(19) United States
(12) Patent Application Publication
(10) Pub. No.: US 2014/0296587 A1
(43) Pub. Date: Oct. 2, 2014

(54) Integrated Process for Increasing Butadiene Production

(71) Applicant: UOP LLC, Des Plaines, IL (US)

(72) Inventors: Andrea G. Bozzano, Northbrook, IL (US); Bipin V. Vora, Naperville, IL (US); Daniel H. Wei, Naperville, IL (US); Steven L. Krupa, Fox River Grove, IL (US)

(73) Assignee: UOP LLC, Des Plaines, IL (US)

(21) Appl. No.: 13/852,473

(22) Filed: Mar. 28, 2013

(51) Int. Cl. C07C 5/48

(52) U.S. Cl. CPC C07C 5/48
USPC 585/252; 585/327

(57) ABSTRACT

A process is present for increasing the yields of 1,3 butadiene. The process includes recovering 1,3 butadiene from a cracking unit that generates a crude C4 stream. The 1,3 butadiene is separated and the remaining C4 process stream components are further reacted and dehydrogenated to generate 1,3 butadiene in a subsequent process stream. The subsequent process stream is recycled to recover the additional 1,3 butadiene.

### FIELD OF THE INVENTION

This invention relates to a process for the production of butadiene. In particular, this is a process for the integration of a butadiene production process into a petrochemical plant.

### BACKGROUND OF THE INVENTION

The use of plastics and rubbers are widespread in today's world. The production of these plastics and rubbers are from the polymerization of monomers which are generally produced from petroleum. The monomers are generated by the breakdown of larger molecules to smaller molecules which can be modified. The monomers are then reacted to generate larger molecules comprising chains of the monomers. An important example of these monomers is light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries. The main source for these materials in present day refining is the steam cracking of petroleum feeds.

Another important monomer is butadiene. Butadiene is a basic chemical component for the production of a range of synthetic rubbers and polymers, as well as the production of precursor chemicals for the production of other polymers. Examples include homopolymerized products such as polybutadiene rubber (PBR), or copolymerized butadiene with other monomers, such as styrene and acrylonitrile. Butadiene is also used in the production of resins such as acrylonitrile butadiene styrene.

Butadiene is typically recovered as a byproduct from the cracking process, wherein the cracking process produces light olefins such as ethylene and propylene. With the increase in demand for rubbers and polymers having the desired properties of these rubbers, an aim to improving butadiene yields from materials in a petrochemical plant will improve the plant economics.

### SUMMARY OF THE INVENTION

The present invention is a process for increasing the butadiene yields from a crude C4 stream. The crude C4 stream is generated by a cracking unit where C4s are a by-product. The process includes a first separation to generate a first byproduct C4 stream. The byproduct C4 stream is passed to a butadiene extraction unit to generate a purified 1,3 butadiene stream and an isobutylene containing C4 stream. The isobutylene containing C4 stream is passed to an MTBE reactor to remove isobutylene, while generating an MTBE product stream, and a second byproduct C4 stream. The second by product C4 stream is passed to a dehydrogenation unit to convert C4s to butadiene in a dehydrogenation process stream. The dehydrogenation process stream is recycled to the butadiene extraction unit.

In another embodiment, the invention comprises passing a raffinate stream from an isobutylene removal unit to generate a process stream comprising n-butane and n-butenes. The process stream is passed to a fractionation unit to generate a bottoms stream comprising n-butane and 2-butene while generating an overhead stream comprising 1-butene. The bottoms stream is passed to an oxidative dehydrogenation unit to produce 1,3 butadiene, and recycling the dehydrogenation process stream to a butadiene extraction unit.

Other objects, advantages and applications of the present invention will become apparent to those skilled in the art from the following detailed description and drawings.

### BRIEF DESCRIPTION OF THE DRAWINGS

FIG. 1 is a first embodiment for increasing the recovery of butadiene from a process that generates a crude C4 stream;

FIG. 2 is a second embodiment of a process for converting the C4 effluent stream from an MTBE reactor to butadiene; and

FIG. 3 is another embodiment for increasing the butadiene yields from a crude C4 stream.

### DETAILED DESCRIPTION OF THE INVENTION

The demand for plastics such as polyethylene and polypropylene has increased substantially, and will continue to increase in the foreseeable future. Due to the increase demand, the increase in demand for the monomers, ethylene and propylene or light olefins, has also increased. This increase in demand has led to improvements in the processes for the production of light olefins. The improvements increase yields from traditional sources, such as naphtha cracking, and from other sources by diverting other hydrocarbon streams for the production of light olefins. For purposes of the present invention, the reference to steam cracking, as used hereinafter, is intended to include any cracking unit, which can be a catalytic cracker, a stream cracker, or a cracking unit for hydrocarbon sources other than naphtha. With increased availability of ethane from associated gas of crude oil production, as well as, from increased recovery of natural gas liquids (NGL) which contains large amounts of ethane, use of ethane as a feed to steam crackers has increased. When ethane is used as a feedstock to a steam cracker, the byproduct C4 stream is significantly reduced. As a result of these changes, the production of byproducts C4s from the cracking process has decreased. An important part of this byproduct is the recovery of butadiene, and through changes in feedstocks for light olefins production, butadiene recovery has been reduced, or is not keeping up with increased demand.

The present invention provides for increasing the recovery of butadiene through an integrated process that generates a butadiene stream through the processing of a byproduct crude C4 stream generated from a naphtha cracker. The present invention provides for the integration with units that can be present in a refinery. For example, the present invention can be used to enhance the production of 1,3-butadiene by integrating with an on-purpose process for the production of 1,3-butadiene. The on-purpose butadiene process stream is combined with the crude C4 stream, which is then passed to the 1,3-butadiene recovery unit. The naphtha cracker can be an existing unit, or a new unit, with this invention added on to increase the recovery of 1,3 butadiene. The present invention is a process including passing a process stream from a cracking unit to a first separation unit to generate a first process C4 stream. The C4 stream is passed to a butadiene extraction unit to generate a 1,3 butadiene stream, and an isobutylene containing C4 stream, also known as Raffinate-I. The extraction unit can also separate out 1,2 butadiene from the first process C4 stream. The isobutylene containing C4 stream is passed to a methyl tertiary butyl ether (MTBE) process unit to generate an MTBE stream and a second process C4 stream, also known as Raffinate-II. The MTBE process unit comprises several components, which are known in the industry, and includes multiple reactors, a reactive distillation column, and other columns for separation of components. The second process C4 stream is passed to a dehydrogenation unit to generate a dehydrogenation process stream that includes 1,3 butadiene. The dehydrogenation process stream is passed to the butadiene extraction unit for an increase in the yield of 1,3 butadiene.

The butadiene extraction is performed using a solvent extraction. An appropriate solvent is a solvent comprising a polar nitrogen compound, or a mixture of polar compounds. Examples of solvents, though not limited to these, include N-methylpyrrolidone (NMP), dimethylformamide (DMF), dimethyl acetamide, and acetonitrile (ACN). A more widely used extractive solvent is NMP.

In the MTBE process unit the isobutylene in the C4 Raffinate-I is reacted with a methanol stream in the MTBE reaction zone or zones to form MTBE. The C4 Raffinate-II stream recovered from the MTBE unit preferably comprises more than 50% n-butenes by weight.

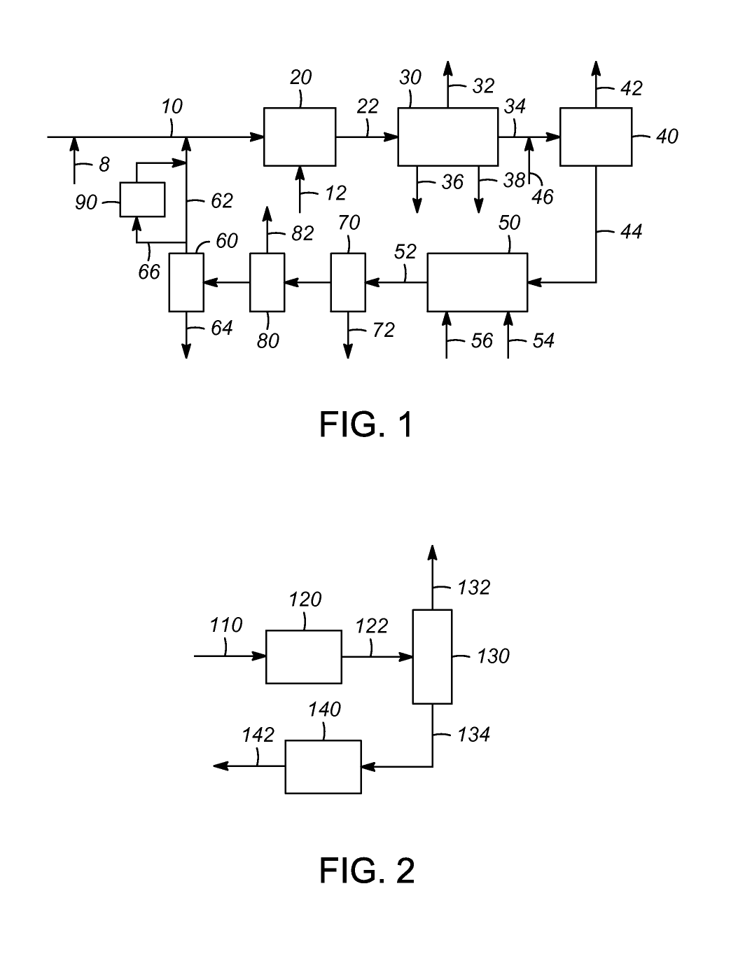

A first embodiment of this process is shown in FIG. 1. The process includes passing a C4 rich stream 10, also known as a crude C4 stream, from a cracking unit to a selective hydrogenation unit 20 to generate a second stream 22 rich in butadienes. The selective hydrogenation unit 20 is for selectively hydrogenating acetylenic compounds, such as vinyl acetylene and ethyl acetylene. The second stream 22 is passed to a butadiene extraction unit 30 to generate a product stream 32 comprising 1,3 butadiene, and an isobutylene containing C4 Raffinate-I stream 34. The isobutylene containing stream 34 is passed to an MTBE process unit 40 to generate an MTBE product stream 42, and a third stream 44 comprising C4 hydrocarbons. The MTBE reactor 40 also receives a methanol stream 46 for reacting with the isobutylene to form MTBE. The third stream 44 is passed to a dehydrogenation unit 50 to dehydrogenate butenes and butanes to butadiene, and generate a dehydrogenation process stream 52. The dehydrogenation process stream 52 is passed to a C4 splitter 60 to generate an overhead stream 62 comprising 1,3-butadiene, and a bottoms stream 64 comprising butanes. The overhead stream 62 is passed to back to be processed and to recover the additional 1,3 butadiene in the butadiene extraction unit 30. In an alternate configuration, an on-purpose butadiene stream 8 is combined with the C4 rich stream from a cracking unit and passed to a common butadiene extraction unit. The on-purpose butadiene stream 8 can be passed to a unit (not shown) for removal of oxygenate impurities, if the stream has not been treated. The oxygenate impurities need to be removed to protect the catalyst used in the selective hydrogenation unit 20.

The overhead stream 62 can first be passed to the selective hydrogenation unit 20 to convert acetylenic compounds. The overhead stream 62 can be passed to an oxygenate removal unit 90 if sufficient oxygenate impurities are not removed prior to the C4 splitter fractionation unit 60. In this case, the overhead stream 62 is passed through 90 via 66 to have oxygenate impurities removed. The oxygenate removal can include washing, fractionation, or an adsorbent unit.

The dehydrogenation unit 50 is preferably an oxidative dehydrogenation unit, and includes passing an oxygen rich stream 54 and a steam stream 56 to the unit 50. Oxidative dehydrogenation has the benefit of generating a relatively high concentration of 1,3 butadiene, while not generating isobutylene or isobutane. The oxidative dehydrogenation process does generate water and oxygenates. The water and a portion of the oxygenates can be removed with a dewatering unit 70 to generate a water rich stream 72 extracted from the dehydrogenation process stream 52. The dehydrogenation process stream 52 can also be passed through a light ends fractionation unit 80 to remove light gases 82, including light hydrocarbons and hydrogen.

The selective hydrogenation unit 20 further includes receiving a hydrogen stream for the hydrogenation of the carbon-carbon triple bonds. The butadiene extraction unit 30 can separate a heavies stream 38, including 1,2 butadiene.

A second embodiment comprises the conversion of a C4 Raffinate-II stream from an MTBE process unit. The C4 Raffinate-II stream comprises C4 compounds that can be converted to 1,3 butadiene. The process is shown in FIG. 2, where a C4 Raffinate-II stream 110 is generated by an MTBE process unit and is passed to a selective hydrogenation unit 120 to generate a first process stream 122. The selective hydrogenation unit 120 converts residual diolefins, typically a small amount of 1,3-butadiene, that will fractionate with the 1-butene. The 1-butene product specifications require a very low level of 1,3 butadiene and its removal is needed to obtain the desired purity of the 1-butene product stream. The first process stream 122 is passed to a fractionation unit 130 to generate an overhead stream 132 and a bottoms stream 134. The overhead stream 132 comprises primarily of 1-butene and isobutane, and can be passed to other units for further processing. 1-Butene is an important co-monomer used with ethylene in the production of polyethylene polymer. The bottoms stream 134 comprises n-butane and 2-butene, and is passed to an oxidative dehydrogenation unit 140 to generate a second process stream 142. The second process stream 142 comprises 1,3 butadiene and is passed to a dewatering and light ends removal as described above and then passed to a butadiene extraction unit. In another embodiment, the stream 142 after dewatering and light ends removal is passed to a fractionation unit, where butadiene rich stream is recovered overhead and passed on to a butadiene extraction unit. The bottoms stream rich in unconverted butenes can be recycle to the dehydrogenation reactor 110. The butadiene extraction unit generates a purified product stream comprising 1,3 butadiene and a C4 Raffinate-I stream including isobutylene.

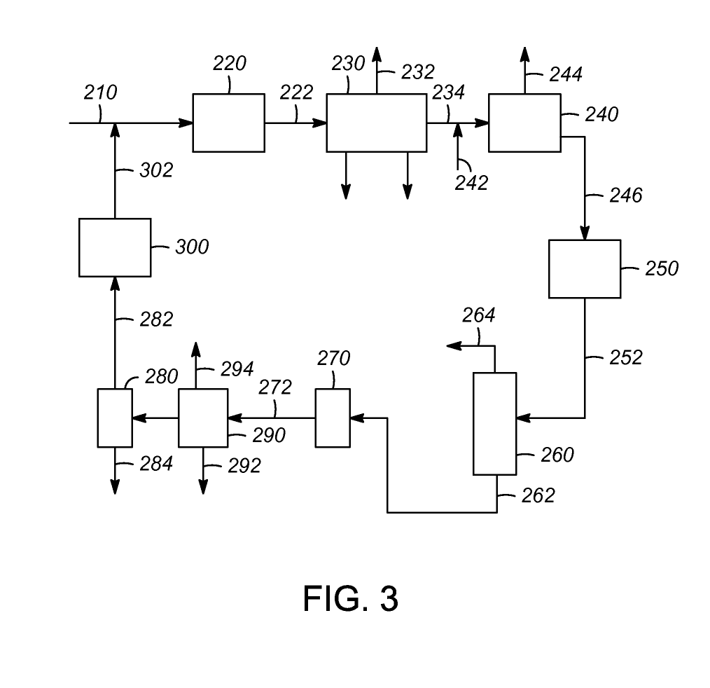

Another embodiment is shown in FIG. 3. The process includes passing a C4 process stream 210 generated by a cracking unit to a selective hydrogenation unit 220 to generate a first process stream 222. The selective hydrogenation unit converts acetylenic compounds to diolefins. The first process stream 222 is passed to a butadiene extraction unit 230 to generate a 1,3 butadiene product stream 232 and a second stream 234 comprising isobutylene. The second stream 234 is passed with a methanol stream 242 to an isobutylene removal unit 240. The isobutylene removal unit 240 generates an product stream 244 and a raffinate stream 246 that includes butanes and butenes.

One example of the isobutylene removal reaction unit 240 can include an MTBE process unit in order to reach a high enough conversion of isobutylene that will enable the high purity recovery of 1-butene after fractionation. In this case, the isobutylene reaction unit 240 can comprise multiple MTBE reactors in series with inter-coolers, or can include one or more MTBE reactors followed by a reactive distillation zone which is integral to the fractionation of C4s and MTBE. The reactive distillation zone overcomes equilibrium constraints found in a single MTBE reactor. The isobutylene reaction unit 240 can include other reactor considerations and configurations for the removal of isobutylene from the second stream 234. Other options can include ethyl tertiary butyl ether (ETBE) reactors, or tertiary butyl alcohol (TBA) reactors. The use of the term isobutylene reaction unit is not intended to be limited to an MTBE reactor, but to a reaction system for the reaction and removal of isobutylenes.

The raffinate stream 246 from an MTBE unit can consist of n-butane and n-butenes with a butene content of nearly 70% by weight. This highly n-butene rich stream stream can be further processed in a dehydrogenation unit to convert the n-butene to butadiene. The converted butadiene stream can be recycled into the butadiene recovery process to increase the butadiene yields.

The raffinate stream 246 is passed to a selective hydrogenation unit 250 to generate a hydrogenated raffinate stream 252. The selective hydrogenation converts residual diolefins, and can perform some isomerization of 1-butene to 2-butene. The amount of 1-butene isomerization can be used as a trade-off of 1-butene production vs. 1,3-butadiene production, giving flexibility to a plant for choice of products. The selective hydrogenation unit 250 converts the residual 1,3-butadiene remaining in the C4 Raffinate-I stream to olefins. The residual amount of 1,3-butadiene in the C4 Raffinate-I stream is typically from 0.2 to 0.5 wt %. The operating conditions and catalyst are chosen to minimize olefin conversion to paraffins. The hydrogenation raffinate stream 252 is a butene rich stream. The hydrogenated raffinate stream 252 is passed to a separation unit 260 to generate a stream 262 rich in 2-butenes. The separation unit 260 also separates out 1-butene as a high value product stream 264. The separation unit 260 can comprise multiple fractionation columns for separating individual components, such as the 1-butene from the hydrogenated raffinate stream 252.

The butenes rich stream 262 is passed to a dehydrogenation unit 270 to generate a dehydrogenation stream 272 rich in 1,3-butadiene. A preferred dehydrogenation unit is an oxidative dehydrogenation unit to limit the production of isobutylenes. The dehydrogenation stream 272 is passed to a C4 splitter 280, after passing through a dewatering unit and a light ends removal unit, to generate an overhead stream 282 comprising butadienes and a bottoms stream 284 rich in unconverted 2-butenes and can be recycled to the dehydrogenation reactor 270. The overhead stream 282 is passed to the feed to the selective hydrogenation unit 220. In an alternative, the overhead stream 282 is passed to an oxygenate removal unit 300 to generate an overhead stream 302 free of oxygenates. The oxygenate free overhead stream 302 is then passed to the selective hydrogenation unit 220.

The oxidative dehydrogenation process generates water and oxygenates, as well as some light gases. The process can include a dewatering unit and degassing unit 290 for removal of water with some oxygenates 292 and removal of light gases 294 comprising C3 and lighter hydrocarbons, and lighter gases.

While the invention has been described with what are presently considered the preferred embodiments, it is to be understood that the invention is not limited to the disclosed embodiments, but it is intended to cover various modifications and equivalent arrangements included within the scope of the appended claims.

1. A process for increasing the production of butadiene, comprising:
passing a process stream from a cracking unit to a first separation unit to generate a first process C4 stream;
passing the first process C4 stream to a butadiene extraction unit to generate a butadiene stream and an isobutylene containing C4 stream;
passing the isobutylene containing C4 stream to a MTBE reactor to generate an MTBE stream and a second process C4 stream;
passing the second process C4 stream to a dehydrogenation unit to generate a dehydrogenation process stream comprising butadienes; and
passing the dehydrogenation process stream to the butadiene extraction unit.

2. The process of claim 1 further comprising passing combining the process stream from a cracking unit with a process stream from an on-purpose butadiene production unit to a common butadiene extraction unit.

3. The process of claim 1 wherein the butadiene extraction unit is a solvent extraction unit.

4. The process of claim 3 wherein the solvent extraction unit uses a solvent compirising a polar nitrogen compound.

5. The process of claim 4 wherein the solvent is selected from the group consisting of NMP(n-methyl-2-pyrrolidone), DMF (dimethylformamide), ACN (acetonitrile), and mixtures thereof.

6. The process of claim 1 further comprising passing methanol to the MTBE unit.

7. The process of claim 1 wherein the second process C4 stream comprises greater than 50% butenes by weight.

8. A process for the production of butadienes comprising:
passing a C4 rich stream from a cracking unit to a selective hydrogenation unit to generate a second stream;
passing the second stream to a butadiene extraction unit to generate at least one butadiene stream and an isobutylene containing C4 stream;
passing the isobutylene containing C4 stream to an MTBE unit to generate an MTBE stream and a third stream comprising C4 hydrocarbons;
passing the third stream to an oxidative dehydrogenation unit to generate an oxidative dehydrogenation process stream comprising butadienes;
passing the process stream to a C4 splitter to generate a bottoms stream comprising butanes, and an overhead stream comprising butadienes; and
passing the overhead stream to the selective hydrogenation unit.

9. The process of claim 8 wherein the selective hydrogenation unit selectively hydrogenates acetylenic compounds.

10. The process of claim 8 further passing the overhead stream to an oxygenate removal unit prior to passing the overhead stream to the selective hydrogenation unit.

11. The process of claim 8 further comprising passing methanol to the MTBE unit.

12. The process of claim 8 further comprising:
passing steam to the oxi-dehydrogenation unit; and
passing a gas stream comprising oxygen to the oxi-dehydrogenation unit.

13. The process of claim 8 further comprising passing the oxi-dehydro process stream to a dewatering unit to generate an oxi-dehydro process stream with reduced water content.

14. The process of claim 13 further comprising passing the oxi-dehydro process stream with reduced water content to a fractionation unit to generate a stream with light ends, and an oxi- dehydro process stream with reduced water content with reduced light hydrocarbons.

15. A process for the production of 1,3-butadiene comprising;
passing a raffinate stream from an MTBE reactor to a selective hydrogenation reactor to generate a first process stream;
passing the first process stream to a fractionation unit to generate an overhead stream comprising 1-butene, and a bottoms stream comprising n-butane and 2-butene; and
passing the bottoms stream to an oxidative dehydrogenation unit to generate a second process stream comprising 1,3-butadiene.

16. The process of claim 15 further comprising passing the second process stream to an butadiene extraction unit to generate at least one stream comprising butadienes and a second stream comprising isobutylene.

17. A process for the production of 1,3-butadiene comprising:
passing a C4 process stream from a cracking unit to a selective hydrogenation unit to generate a first process stream;
passing the first process stream to a butadiene extraction unit to generate at least one butadiene stream and a second stream comprising isobutylene;
passing the second stream to an isobutylene removal unit to generate an isobutylene reaction product stream and a raffinate stream comprising butanes and butenes;
passing the raffinate stream to a selective hydrogenation reactor to generate a hydrogenated raffinate stream;
passing the hydrogenated raffinate stream to a separation unit to generate a butenes rich stream;
passing the butenes rich stream to a dehydrogenation unit to generate a dehydrogenation stream comprising butadienes;
passing the dehydrogenation stream to a C4 splitter to generate an overhead stream rich in 1,3-butadiene and a bottoms stream comprising nC4s; and
passing the overhead stream to the selective hydrogenation unit.

18. The process of claim 17 wherein the separation unit comprises at least one fractionation column and generates a 1-butene stream and a 2-butene rich stream.

19. The process of claim 17 further comprising dewatering the dehydrogenation stream.

20. The process of claim 17 further comprising removing C3-hydrocarbons and light gases from the dehydrogenation stream.
